(19) United States
(12) Patent Application Publication
(10) Pub. No.: US 2005/0176621 A1
(43) Pub. Date: Aug. 11, 2005

(54) Crystalline compositions for controlling blood glucose

(76) Inventors: Mark Laurence Brader, Indianapolis, IN (US); Sharon Ruth Myers, Indianapolis, IN (US); Muppalla Sukumar, Indianapolis, IN (US)

Correspondence Address: ELI LILLY AND COMPANY;PATENT DIVISION, P.O. BOX 6288, INDIANAPOLIS, IN, 46206-6288, US

(21) Appl. No.: 10/600,031

(22) Filed: Jun. 20, 2003

Related U.S. Application Data
(63) Continuation-in-part of application No. PCT/US02/37602, filed on Dec. 12, 2002.
(60) Provisional application No. 60/342,321, filed on Dec. 19, 2001.

(51) Int. Cl. A61K 38/00

(52) U.S. Cl. 514/2

(57) ABSTRACT

The present invention relates to insulin crystals formed from zinc, protamine, a hexamer-stabilizing compound, and a polypeptide selected from the group consisting of insulin, an insulin analog, and a derivatized insulin. The crystals are suitable for administering to a patient for control of blood glucose levels. The crystals have been derived from the neutral protamine Hagedorn (NPH) form in a process utilizing precisely determined protamine concentrations and fortification of NPH crystals formed at a first lower concentration of protamine to achieve a second higher concentration of protamine.

### CROSS-REFERENCE TO RELATED APPLICATIONS

This application is a continuation-in-part of international patent application no. PCT/US02/37602, filed Dec. 12, 2002 (pending), which claims the benefit of U.S. Provisional Application 60/342,321, filed Dec. 19, 2001, both of which are incorporated by reference.

### FIELD OF THE INVENTION

This invention is in the field of human medicine. More particularly, this invention is in the field of pharmaceutical treatment of the diseases of diabetes and hyperglycemia.

### BACKGROUND OF THE INVENTION

Effective insulin therapy for people with diabetes generally involves the combined use of two types of exogenous insulin formulations: a rapid acting meal time insulin provided by injections to dispose of the meal-related blood glucose surge, and a long-acting, so-called, basal insulin, administered by injection once or twice daily to control blood glucose levels between meals. Insulin NPH (Neutral Protamine Hagedorn) is the most widely-used basal insulin preparation, constituting from 50 to 70 percent of the insulin used worldwide. It is a suspension of a crystalline complex of insulin, zinc, protamine, and one or more phenolic preservatives.

Therapy using currently-available NPH insulin preparations fails to provide the ideal “flat” pharmacokinetics necessary to maintain optimal fasting blood glucose for an extended period of time between meals. Consequently, treatment with NPH insulin can result in undesirably high levels of insulin in the blood, which may cause life-threatening hypoglycemia. In addition to failing to provide an ideal flat pharmacokinetic profile, the duration of action of NPH insulin also is not ideal. In particular, a major problem with NPH therapy is the “dawn phenomenon” which is hyperglycemia that results from the loss of effective glucose control overnight while the patient is sleeping.

Protamine zinc insulin (PZI) is a basal insulin that is similar to NPH, but contains higher levels of protamine and zinc than NPH. PZI preparations may be made as intermediate-acting amorphous precipitates or long-acting crystalline material. PZI, however, is not an ideal basal insulin pharmaceutical because it is not mixable with a soluble meal-time insulin, and the high zinc and protamine can cause irritation or reaction at the site of administration.

Human insulin ultralente is a microcrystalline preparation of insulin having higher levels of zinc than NPH, and not having either protamine or a phenolic preservative incorporated into the microcrystal. Human ultralente preparations provide moderate time action that is not suitably flat, and they do not form stable mixtures with insulin. Furthermore, the ultralente microcrystals are difficult to resuspend.

Thus, there remains a need to identify insulin preparations that have flatter and longer time action than NPH insulin, that are mixable with soluble, meal-time insulins, that can be readily resuspended, and that do not pose risk of irritation or reaction at the site of administration.

### SUMMARY OF THE INVENTION

The present invention provides non-adsorbed insulin crystals comprising zinc, protamine, a hexamer-stabilizing compound, and a polypeptide selected from the group consisting of insulin, an insulin analog, and a derivatized insulin, wherein less than 2% of the polypeptide is present on the non-adsorbed insulin crystals as adsorbed insulin.

This invention further provides a method of making non-adsorbed insulin crystals which comprises: crystallizing zinc, a first concentration of protamine, a hexamer-stabilizing compound and a polypeptide selected from the group consisting of insulin, an insulin analog, and a derivatized insulin, to form adsorbed insulin crystals; and combining the adsorbed insulin crystals with protamine so as to achieve a second, higher concentration of protamine to form the non-adsorbed insulin crystals, wherein less than 2% of said polypeptide is present on said non-adsorbed insulin crystals as adsorbed polypeptide.

In addition, the present invention provides a method of treating diabetes mellitus by administering a composition comprising non-adsorbed insulin crystals, wherein the non-adsorbed insulin crystals contain zinc, protamine, a hexamer stabilizing compound, and a polypeptide selected from the group consisting of insulin, an insulin analog, and a derivatized insulin.

### BRIEF DESCRIPTION OF THE DRAWINGS

FIG. 1. Scanning electron microscopy images of a preparation of recombinant human insulin NPH crystals (Humulin® N) (FIG. 1A) and a preparation of recombinant human insulin non-adsorbed insulin crystals (FIG. 1B). Overlaid on each image is a 5 micron scale bar.

### DESCRIPTION OF THE INVENTION

As used herein, “non-adsorbed insulin crystals” refers to crystals of insulin, zinc, protamine, and a hexamer-stabilizing compound that contain little to no insulin adsorbed to the surface of the crystal. In a preparation of non-adsorbed insulin crystals, 0 to less than 2% of the insulin associated with the crystals will be adsorbed to the surfaces of the crystals. More preferably, 0 to 1%, and even more preferably 0 to 0.2% of the insulin associated with the non-adsorbed insulin crystals will be adsorbed to the surfaces of the crystals. Following formation of non-adsorbed insulin crystals, the remaining solution supernatant contains between zero to about 0.2% soluble insulin that is not incorporated into or associated with crystals.

The term “adsorbed insulin crystals” refers to crystals of insulin, zinc, protamine, and a hexamer-stabilizing compound that contain insulin adsorbed to the surface of the crystal. Conventional preparations of NPH insulin provide adsorbed insulin crystals. In adsorbed insulin crystal preparations, typically 3% to 5% of the insulin associated with the crystals is adsorbed to the surfaces of the crystals. Following formation of adsorbed insulin crystals, the remaining solution supernatant contains between about 0.4% to about 0.9% soluble insulin that is not incorporated into or associated with crystals.

As used herein “adsorbed insulin” refers to insulin that is adsorbed to insulin crystals in a manner in which the adsorbed insulin is associated with insulin crystals but is not itself in a crystalline state. The term “adsorbed polypeptide” as used herein refers to insulin, an insulin derivative, or an insulin analog that is adsorbed to crystals of the insulin, insulin derivative, or insulin analog such that the adsorbed polypeptide is associated with the crystals but is not itself in a crystalline state.

The term “amorphous precipitate” refers to insoluble material that is not crystalline in form. The person of ordinary skill can distinguish crystals from amorphous precipitate.

The term “immediately available insulin” refers to either soluble insulin in solution or insulin that is adsorbed to the surface of an insulin crystal. The amount of immediately available insulin that is adsorbed to a preparation of insulin crystals may be determined by the immediately available insulin assay, which is described herein.

The term “immediately available insulin assay” (IAIA) refers to an assay used to determine the amount of insulin that is adsorbed to insulin crystals.

“NPH insulin” refers to the “Neutral Protamine Hagedorn” preparation of insulin. Synonyms include human insulin NPH and insulin NPH, among many others. Humulin® N is a commercial preparation of NPH insulin. A related term is “NPL” which refers to an NPH-like preparation of LysB28,ProB29-human insulin analog. The meaning of these terms, and the methods for preparing them will be familiar to the person of ordinary skill in the insulin formulation art.

The term “crystal” as used herein means a solid that is comprised primarily of insoluble matter in a crystalline state. The insoluble “crystal” solid is typically greater than 90% matter in a crystalline state, with remaining insoluble matter being amorphous precipitate. The amounts of crystalline and amorphous precipitate in crystal preparations are typically determined by microscopic examination. The term “crystalline” refers to the state of being a crystal.

The term “crystallizing” as used herein refers to the process of forming insulin crystals.

The individual crystals are predominantly of a single crystallographic composition and are of a microscopic size, typically of longest dimension within the range 0.5 micron to 15 microns. Preferably, the length of the longest dimension of the crystals is between 0.5-10 microns, more preferably between 0.5-5 microns, and even more preferably between 0.5-3 microns. One of ordinary skill in the art will recognize that these ranges refer to crystals in which the mean crystal length and the majority of the associated distribution of lengths will lie within the ranges. Accordingly, a preparation of crystals of the present invention will contain crystals in which at least 95%, more preferably 97%, and more preferably still 99% of the crystals have a longest dimension within a given preferred range.

The term “irregular morphology” is a characterization of crystals whose morphology, as determined by microscopic examination, is not readily classified into any of the well-known crystal types, is not a single type of crystal morphology, or is not readily determinable because the size of the crystals is too small for certain classification.

As used herein, the term “combining” means to combine two or more components to form a mixture of the components.

The term “insulin” as used herein, refers to human insulin, whose amino acid sequence and special structure are well-known.

The term “derivatized insulin” refers to a polypeptide selected from the group consisting of derivatized insulin, a derivatized insulin analog, derivatized proinsulin, and a derivatized proinsulin analog that is derivatized by a functional group such that the derivatized protein is less soluble in an aqueous solvent, is more lipophilic than un-derivatized insulin, or produces a complex with zinc and protamine that are less soluble than the corresponding complex with the un-derivatized protein. The determination of either the solubility or lipophilicity of insulins and derivatized insulins is well-known to the skilled person. The solubility of derivatized insulin and insulin in complexes with zinc and protamine can be readily determined by well-known procedures [Graham and Pomeroy, J. Pharm. Pharmacol. 36: 427-430 (1983), as modified in DeFelippis, M. R. and Frank, B., EP 735,048].

Many examples of such derivatized proteins are known in the art, including benzoyl, p-tolyl-sulfonamide carbonyl, and indolyl derivatives of insulin and insulin analogs [Havelund, S., et al., WO95/07931, published 23 Mar. 1995]; alkyloxycarbonyl derivatives of insulin [Geiger, R., et al., U.S. Pat. No. 3,684,791, issued 15 Aug. 1972; Brandenberg, D., et al., U.S. Pat. No. 3,907,763, issued 23 Sep. 1975]; aryloxycarbonyl derivatives of insulin [Brandenberg, D., et al., U.S. Pat. No. 3,907,763, issued 23 Sep. 1975]; alkylcarbamyl derivatives [Smyth, D. G., U.S. Pat. No. 3,864,325, issued 4 Feb. 1975; Lindsay, D. G., et al., U.S. Pat. No. 3,950,517, issued 13 Apr. 1976]; carbamyl, O-acetyl derivatives of insulin [Smyth, D. G., U.S. Pat. No. 3,864,325 issued 4 Feb. 1975]; cross-linked, alkyl dicarboxyl derivatives [Brandenberg, D., et al., U.S. Pat. No. 3,907,763, issued 23 Sep. 1975]; N-carbamyl, O-acetylated insulin derivatives [Smyth, D. G., U.S. Pat. No. 3,868,356, issued 25 Feb. 1975]; various O-alkyl esters [Markussen, J., U.S. Pat. No. 4,343,898, issued 10 Aug. 1982; Morihara, K., et al., U.S. Pat. No. 4,400,465, issued 23 Aug. 1983; Morihara, K., et al., U.S. Pat. No. 4,401,757, issued 30 Aug. 1983; Markussen, J., U.S. Pat. No. 4,489,159, issued 18 Dec. 1984; Obermeier, R., et al., U.S. Pat. No. 4,601,852, issued 22 Jul. 1986; and Andresen, F. H., et al., U.S. Pat. No. 4,601,979, issued 22 Jul. 1986]; alkylamide derivatives of insulin [Balschmidt, P., et al., U.S. Pat. No. 5,430,016, issued 4 Jul. 1995]; various other derivatives of insulin [Lindsay, D. G., U.S. Pat. No. 3,869,437, issued 4 Mar. 1975]; and the fatty acid-acylated insulins that are described herein.

The term “acylated insulin” as used herein refers to a derivatized polypeptide selected from the group consisting of insulin, an insulin analog, proinsulin, and a proinsulin analog that is acylated with an organic acid moiety that is bonded to the insulin through an amide bond formed between the acid group of an organic acid compound and an amino group of the insulin. In general, the amino group may be the α-amino group of an N-terminal amino acid of the insulin, or may be the ε-amino group of a Lys residue of the insulin. An acylated insulin may be acylated at one, two, or three of the three amino groups that are present in insulin and in most insulin analogs. The organic acid compound may be, for example, a fatty acid, an aromatic acid, or any other organic compound having a carboxylic acid group that will form an amide bond with an amino group of a protein, and that will lower the aqueous solubility, raise the lipophilicity, or decrease the solubility of zinc/protamine complexes of the derivatized insulin compared with the un-derivatized insulin.

The term “fatty acid-acylated insulin” refers to an acylated protein selected from the group consisting of insulin, insulin analogs, and proinsulins that is acylated with a fatty acid that is bonded to the insulin through an amide bond formed between the acid group of the fatty acid and an amino group of the protein. In general, the amino group may be the α-amino group of an N-terminal amino acid of the insulin, or may be the ε-amino group of a Lys residue of the insulin. A fatty acid-acylated protein may be acylated at one, two, or three of the three amino groups that are present in insulin and in most insulin analogs. Fatty acid-acylated insulin is disclosed in a Japanese patent application 1-254,699. See also, Hashimoto, M., et al., Pharmaceutical Research, 6: 171-176 (1989), and Lindsay, D. G., et al., Biochemical J. 121: 737-745 (1971). Further disclosure of fatty acid-acylated insulins and fatty acylated insulin analogs, and of methods for their synthesis, is found in Baker, J. C., et al, U.S. Ser. No. 08/342,931, filed 17 Nov. 1994 and issued as U.S. Pat. No. 5,693,609, 2 Dec. 1997; Havelund, S., et al., WO95/07931, published 23 Mar. 1995, and a corresponding U.S. Pat. No. 5,750,497, 12 May 1998; and Jonassen, I., et al., WO96/29342, published 26 Sep. 1996.

The term “fatty acid-acylated insulin” includes pharmaceutically acceptable salts and complexes of fatty acid-acylated insulins. The term “fatty acid-acylated insulin” also includes preparations of acylated insulins wherein the population of acylated insulin molecules is homogeneous with respect to the site or sites of acylation. For example, Nε-mono-acylated insulin, B1-Nα-mono-acylated insulin, A1-Nα-mono-acylated insulin, A1,B1-Nα-di-acylated insulin, Nε,A1-Nα,di-acylated insulin, Nε,B1-Nα,di-acylated insulin, and Nε,A1,B1-Nα,tri-acylated insulin are all encompassed within the term “fatty acid-acylated insulin” for the purpose of the present invention. The term also refers to preparations wherein the population of acylated protein molecules has heterogeneous acylation. In the latter case, the term “fatty acid-acylated insulin” includes mixtures of mono-acylated and di-acylated insulins, mixtures of mono-acylated and tri-acylated insulins, mixtures of di-acylated and tri-acylated insulins, and mixtures of mono-acylated, di-acylated, and tri-acylated insulins.

The term “insulin analog” means proteins that have an A-chain and a B-chain that have substantially the same amino acid sequences as the A-chain and B-chain of human insulin, respectively, but differ from the A-chain and B-chain of human insulin by having one or more amino acid deletions, one or more amino acid replacements, and/or one or more amino acid additions that do not destroy the insulin activity of the insulin analog.

“Animal insulins” are an example of insulin analogs. Four such animal insulins are rabbit, pork, beef, and sheep insulin. The amino acid substitutions that distinguish these animal insulins from human insulin are presented below for the reader's convenience.

Amino Acid Position

| | A8 | A9 | A10 | B30 |
|---|---|---|---|---|
| human insulin | Thr | Ser | Ile | Thr |
| rabbit insulin | Thr | Ser | Ile | Ser |
| pork insulin | Thr | Ser | Ile | Ala |
| beef insulin | Ala | Ser | Val | Ala |
| sheep insulin | Ala | Gly | Val | Ala |

A “rapid-acting insulin analog” provides a hypoglycemic effect that (a) begins sooner after subcutaneous administration than human insulin, and/or (b) exhibits a shorter duration of action than human insulin after subcutaneous administration. B28LysB29Pro-insulin (so-called “lispro” insulin) is a rapid-acting insulin analog, in which the Pro at position 28 of the wild-type insulin B-chain (Seq. ID No. 4) and the Lys at position 29 of the wild-type insulin B-chain (Seq. ID No. 4) have been switched. See, for example, U.S. Pat. Nos. 5,504,188 and 5,700,662. Another rapid-acting insulin analog is B28Asp-insulin, in which the wild-type Pro at position 28 of the B-chain has been replaced by Asp. See U.S. Pat. No. 5,547,930. Another rapid-acting insulin analog is B3LysB29Glu-insulin. See U.S. Pat. No. 6,221,633.

Another group of insulin analogs for use in the present invention are those wherein the isoelectric point of the insulin analog is between about 7.0 and about 8.0. These analogs are referred to as “pi-shifted insulin analogs.” Examples of such insulin analogs include the analogs disclosed in PCT/US02/37601, and ArgB31,ArgB32-human insulin, GlyA21,ArgB31,ArgB32-human insulin, ArgA0,ArgB31,ArgB32-human insulin, and ArgA0,GlyA21,ArgB31,ArgB32-human insulin.

Another group of insulin analogs consists of insulin analogs that have one or more amino acid deletions that do not significantly disrupt the activity of the molecule. This group of insulin analogs is designated herein as “deletion analogs.” For example, insulin analogs with deletion of one or more amino acids at positions B1-B3 are active. Likewise, insulin analogs with deletion of one or more amino acids at positions B28-B30 are active. Examples of “deletion analogs” include des(B30)-human insulin, desPhe(B1)-human insulin, des(B27)-human insulin, des(B28-B30)-human insulin, and des(B1-B3)-human insulin.

An insulin analog may be insulin or an insulin analog that has one or more of its amidated residues replaced with other amino acids for the sake of chemical stability. For example, Asn or Gln may be replaced with a non-amidated amino acid. Preferred amino acid replacements for Asn or Gln are Gly, Ser, Thr, Asp or Glu. It is preferred to replace one or more Asn residues. In particular, AsnA18, AsnA21, or AsnB3, or any combination of those residues may be replaced by Gly, Asp, or Glu, for example. Also, GlnA15 or GlnB4, or both, may be replaced by either Asp or Glu. Preferred replacements are Asp at B21, and Asp at B3. Also preferred are replacements that do not change the charge on the protein molecule, so that replacement of Asn or Gln with neutral amino acids is also preferred. Examples of such analogs can be found in U.S. Pat. No. 5,008,241 and U.S. Pat. No. 5,656,722.

The term “proinsulin” means a single-chain peptide molecule that is a precursor of insulin. Proinsulin may be converted to insulin or to an insulin analog by chemical or, preferably, enzyme-catalyzed reactions. In proinsulin, proper disulfide bonds are formed as described herein. Proinsulin may have the formula X-B-C-A-Y or may have the formula X-A-C-B-Y, wherein X is hydrogen or is a peptide of from 1 to about 100 amino acids that has either Lys or Arg at its C-terminal amino acid, Y is hydroxy, or is a peptide of from 1 to about 100 amino acids that has either Lys or Arg at its N-terminal amino acid, A is the A-chain of insulin or the A-chain of an insulin analog, C is a peptide of from 1 to about 35 amino acids, none of which is cysteine, wherein the C-terminal amino acid is Lys or Arg, and B is the B-chain of insulin or the B-chain of an insulin analog.

A “pharmaceutically acceptable salt” means a salt formed between any one or more of the charged groups in a protein and any one or more pharmaceutically acceptable, non-toxic cations or anions.

The verb “acylate” means to form the amide bond between a fatty acid and an amino group of a protein. A protein is “acylated” when one or more of its amino groups is combined in an amide bond with the acid group of a fatty acid.

The term “fatty acid” means a saturated or unsaturated, straight chain or branched chain fatty acid, having from one to eighteen carbon atoms.

The term “C1 to C18 fatty acid” refers to a saturated, straight chain or branched chain fatty acid having from one to eighteen carbon atoms.

The term “protamine” refers to a mixture of strongly basic proteins obtained from fish sperm. The average molecular weight of the proteins in protamine is about 4,200 [Hoffmann, J. A., et al., Protein Expression and Purification, 1:127-133 (1990)]. “Protamine” can refer to a relatively salt-free preparation of the proteins, often called “protamine base.” Protamine also refers to preparations comprised of salts of the proteins. Commercial preparations vary widely in their salt content.

Protamines are well-known to those skilled in the insulin art and are currently incorporated into NPH insulin products. A pure fraction of protamine is operable in the present invention, as well as mixtures of protamines. Commercial preparations of protamine, however, are typically not homogeneous with respect to the proteins present. These are nevertheless operative in the present invention. Protamine comprised of protamine base is operative in the present invention, as are protamine preparations comprised of salts of protamine, and those that are mixtures of protamine base and protamine salts. Protamine sulfate is a frequently used protamine salt. All mass ratios referring to protamine are given with respect to protamine free base. The person of ordinary skill can determine the amount of other protamine preparations that would meet a particular mass ratio referring to protamine.

The term “suspension” refers to a mixture of a liquid phase and a solid phase that consists of insoluble or sparingly soluble particles that are larger than colloidal size. Mixtures of NPH-like crystals and an aqueous solvent form suspensions. The term “suspension formulation” means a pharmaceutical composition wherein an active agent is present in a solid phase, for example, a crystalline solid which is finely dispersed in an aqueous solvent. The finely dispersed solid is such that it may be suspended in a fairly uniform manner throughout the aqueous solvent by the action of gently agitating the mixture, thus providing a reasonably uniform suspension from which a dosage volume may be extracted. Examples of commercially available insulin suspension formulations include, for example, NPH, PZI, and ultralente.

The term “liquid solution” as used herein refers to a solution that contains no insoluble crystals or precipitates.

The term “aqueous solvent” refers to a liquid solvent that contains water. An aqueous solvent system may be comprised solely of water, may be comprised of water plus one or more miscible solvents, and may contain solutes. The more commonly-used miscible solvents are the short-chain organic alcohols, such as, methanol, ethanol, propanol, short-chain ketones, such as acetone, and polyalcohols, such as glycerol.

An “isotonicity agent” is a compound that is physiologically tolerated and imparts a suitable tonicity to a formulation to prevent the net flow of water across cell membranes that are in contact with an administered formulation. Glycerol, which is also known as glycerin, is commonly used as an isotonicity agent. Other isotonicity agents include salts, e.g., sodium chloride, and monosaccharides, e.g., dextrose and lactose.

The compositions of the present invention contain a hexamer-stabilizing compound. The term “hexamer-stabilizing compound” refers to a non-proteinaceous, small molecular weight compound that stabilizes the insulin in a hexameric aggregation state. Phenolic compounds, particularly phenolic preservatives, are the best known stabilizing compounds for insulin and insulin derivatives. Examples of hexamer-stabilizing agents include: various phenolic compounds, phenolic preservatives, resorcinol, 4′-hydroxyacetanilide, 4-hydroxybenzamide, and 2,7-dihyroxynaphthalene.

The term “preservative” refers to a compound added to a pharmaceutical formulation to act as an anti-microbial agent. A parenteral formulation must meet guidelines for preservative effectiveness to be a commercially viable multi-use product. Among preservatives known in the art as being effective and acceptable in parenteral formulations are benzalkonium chloride, benzethonium, chlorohexidine, phenol, m-cresol, benzyl alcohol, methylparaben, chlorobutanol, o-cresol, p-cresol, chlorocresol, phenylmercuric nitrate, thimerosal, benzoic acid, and various mixtures thereof. See, e.g., Wallhausser, K.-H., Develop. Biol. Standard, 24:9-28 (1974) (S. Krager, Basel). The preservative used in formulations of the present invention may be the same as, or different from the hexamer-stabilizing compound.

The term “phenolic preservative” includes the compounds phenol, m-cresol, o-cresol, p-cresol, chlorocresol, methylparaben, and mixtures thereof. Certain phenolic preservatives, such as phenol and m-cresol, are known to bind to insulin-like molecules and thereby to induce conformational changes that increase either physical or chemical stability, or both [Birnbaum, D. T., et al., Pharmaceutical. Res. 14:25-36 (1997); Rahuel-Clermont, S., et al., Biochemistry 36:5837-5845 (1997)].

The term “buffer” or “pharmaceutically acceptable buffer” refers to a compound that is known to be safe for use in insulin formulations and that has the effect of controlling the pH of the formulation at the pH desired for the formulation. The pH of the formulations of the present invention is from about 6.0 to about 8.0. Preferably the formulations of the present invention have a pH between about 6.8 and about 7.8. Pharmaceutically acceptable buffers for controlling pH at a moderately acidic pH to a moderately basic pH include such compounds as phosphate, acetate, citrate, arginine, TRIS, and histidine. “TRIS” refers to 2-amino-2-hydroxymethyl-1,3,-propanediol, and to any pharmacologically acceptable salt thereof. The free base and the hydrochloride form are two common forms of TRIS. TRIS is also known in the art as trimethylol aminomethane, tromethamine, and tris(hydroxymethyl)aminomethane. Other buffers that are pharmaceutically acceptable, and that are suitable for controlling pH at the desired level are known to the chemist of ordinary skill.

The term “administer” means to introduce a formulation of the present invention into the body of a patient in need thereof to treat a disease or condition.

The term “treating” refers to the management and care of a patient having diabetes or hyperglycemia, or other condition for which insulin administration is indicated for the purpose of combating or alleviating symptoms and complications of those conditions. Treating includes administering a formulation of present invention to prevent the onset of the symptoms or complications, alleviating the symptoms or complications, or eliminating the disease, condition, or disorder.

A clinically normal fasting plasma glucose level is 70-110 mg/dl. A clinically normal postprandial plasma glucose level is less than 140 mg/dl. “Sufficient to regulate blood glucose in a subject” means that administration of an insulin molecule results in a clinically normal fasting plasma glucose level.

As is well-known to those of ordinary skill in the art, insulin effect can be quantified using the “glucose clamp” technique, in which the amount of exogenous glucose required over time to maintain a predetermined plasma glucose level is used as a measure of the magnitude and duration of an insulin effect caused by an insulin molecule. For example, see Burke et al., Diabetes Research, 4:163-167 (1987). Typically, in a glucose clamp investigation, glucose is infused intravenously. If an insulin molecule causes a decrease in plasma glucose level, the glucose infusion rate is increased, such that the predetermined plasma glucose level is maintained. When the insulin molecule effect diminishes, the glucose infusion rate is decreased, such that the predetermined plasma glucose level is maintained.

“Insulin effect” means that in a glucose clamp investigation, administration of an insulin molecule requires that the rate of intravenous blood glucose administration be increased in order to maintain a predetermined plasma glucose level in the subject for the duration of the glucose clamp experiment. In one preferred embodiment, the predetermined glucose level is a fasting plasma glucose level. In another preferred embodiment, the predetermined glucose level is a postprandial plasma glucose level.

An insulin molecule or formulation has a “protracted duration of action” if the insulin molecule or formulation provides an insulin effect in hyperglycemic, e.g., diabetic, patients that lasts longer than regular human insulin. Preferably the insulin molecule or formulation provides an insulin effect for from about 8 hours to about 24 hours after a single administration of the insulin molecule or formulation. More preferably the insulin effect lasts from about 10 hours to about 24 hours. Even more preferably, the effect lasts from about 12 hours to about 24 hours. Still more preferably, the effect lasts from about 16 hours to about 24 hours. Most preferably, the effect lasts from about 20 hours to about 24 hours.

An insulin molecule or formulation has a “basal insulin effect” if the insulin molecule or formulation provides a glucose lowering effect in subjects that lasts about 24 hours after a single administration of the insulin molecule or formulation.

The present invention provides insoluble non-adsorbed insulin crystals that have properties well suited for a basal insulin and superior to NPH. A goal of basal insulin therapy is to mimic the pattern of endogenous insulin secretion in normal individuals, which requires a sustained delivery of insulin to regulate hepatic glucose output for maintaining optimal fasting blood glucose. An ideal basal insulin will provide an extended and “flat” time action, in which it will control blood glucose levels for at least 12 hours, and preferably for 24 hours or more, without significant risk of hypoglycemia. As is well-known in the art, time action of insulin may be determined by the glucose clamp technique.

Rather than having a flat time action of an ideal basal insulin, the insulin activity of NPH fluctuates. In particular, the time action of NPH has a peak of insulin activity following administration, such that the insulin activity over the initial four hour interval of therapy is typically greater than that of any subsequent four hour interval of therapy, with the time action profile of NPH typically extending out to about 13-16 hours. Measuring the insulin activity of NPH with the glucose clamp technique, administration of NPH therefore results in a glucodynamic peak over the first 4 hours post administration of NPH as compared to hours 4-16 post administration. The mean maximum rate (Rₘₐₓ) of glucose infusion for a four hour period in the first 0 to 4 hours is typically about 1 to about 10 times greater than that for any other four hour interval between hours 4 to 22 hours post administration.

The insulin crystals of the present invention provide for a flatter profile of blood glucose control than does NPH. In particular, the insulin crystals disclosed herein significantly decrease the initial peak of activity observed with NPH, such that there is little to no initial glucodynamic peak in the time action following administration of these insulin crystals. Specifically, the mean Rₘₐₓ for glucose infusion (as determined by the glucose clamp technique) during the first four hours following administration is preferably no greater than 1.75 times than that experienced during any other four hour interval between 4 to 22 hours. This first four hour Rₘₐₓ is more preferably no greater than 1.5 times, and even more preferably no greater than 1.25 times that of any other four hour interval. Thus, the insulin crystals of the present invention are herein referred to as “peakless” or, alternatively, as possessing a smaller glucodynamic peak than NPH.

Without being bound by any particular theory, Applicants believe the insulin crystals of the present invention are peakless due to the absence of non-crystalline, adsorbed insulin on the crystal. The insulin crystals of the present invention are therefore referred to as “non-adsorbed insulin crystals.” In contrast, NPH crystals are referred to as “adsorbed insulin crystals,” since they are believed to have non-crystalline insulin adsorbed on their surface. This adsorbed, non-crystalline insulin is referred to as “immediately available insulin.”

The presence or absence of adsorbed insulin on non-soluble insulin preparations has been determined by the use of an immediately available insulins assay (IAIA). In this assay, a preparation of insoluble insulin, such as NPH or the non-adsorbed insulin crystals of the present invention, is suspended in buffer, filtered, and then the filtrate is analyzed by HPLC to determine the presence of insulin. More specifically, a preparation of insoluble insulin is first resuspended by gentle agitation. A volume of this suspension is then combined with an equal volume of 0.1 M Tris buffer, pH 8.20 (pH at 25° C.) and allowed to stand at room temperature for 10 minutes. The mixture is then filtered through a 0.2 micron low protein binding filter, such as an Acrodisc 13 mm HT Tuffryn membrane. It is important to account for the loss of protein due to binding to the filter, so that erroneously low values of immediately available insulin are not obtained. A person skilled in the art will be able to determine how to account for the loss of protein during filtration, e.g. by passing and discarding a certain portion of the analyte so as to saturate the filter and analyzing a subsequent sample passed through the same filter. The filtrate is then analyzed by HPLC.

Analysis of NPH and non-adsorbed insulin crystals using the IAIA has shown that NPH crystals have from about 3% to about 5% adsorbed insulin, whereas the non-adsorbed insulin crystals of the present invention have 0% to less than 2% adsorbed insulin. The difference between the amount of measurable insulin for the NPH and non-adsorbed insulin crystals is believed by Applicants to reflect the presence of non-crystalline insulin that is associated with insulin crystals by being adsorbed to the insulin crystals, rather than by being part of the crystalline matrix. Commensurately, Applicants believe this adsorbed insulin is immediately available for uptake in the bloodstream upon administration of NPH relative to the truly crystalline portion of the insulin crystals. Thus, the initial peak of insulin activity observed with NPH is attributable to immediately available insulin, whereas the non-adsorbed insulin crystals contain little to no such insulin, and therefore render a flatter time action than NPH.

In addition to being peakless as compared to NPH, the non-adsorbed insulin crystals may also possess a longer time action duration than does NPH. This increased duration of time action, as measured by glucose clamp, will typically result in a mean glucose infusion rate for the non-adsorbed insulin crystals that is greater than that for NPH at later time periods following administration of crystals. In particular, the non-adsorbed crystals will have a mean glucose infusion rate that is greater than that obtained with NPH at 16 to 18, preferably 18 to 20, and more preferably 20 to 22 hours following administration of insulin crystals.

It is known in the art that mixing preparations of NPH crystalline insulin and soluble insulin measurably reduces soluble insulin in the resulting suspension due to adsorption of soluble insulin to the crystalline insulin (Dodd et al., Pharm. Res., 12:60-68 (1995)). However, prior to the present invention, it was not recognized that preparations of NPH would themselves contain adsorbed insulin crystals, and that such adsorbed insulin is a measure that reflects the glucodynamic peak in the first 0 to 4 hours after administration of NPH. Significantly, Applicants have discovered zinc protamine insulin crystals that have little to no insulin adsorbed to the crystalline matrix, as well as how to prepare these non-adsorbed insulin crystals.

The non-adsorbed insulin crystals of the present invention are derived from initially formed, adsorbed insulin crystals, such as NPH, in a two step process, as detailed herein.

Adsorbed insulin crystals are initially formed at a first, precise protamine concentration which is critical for crystallization. If the concentration is too low, it will not be sufficient to drive crystallization of the insulin towards completion, which yields most (greater than 90% or more) of the insulin crystalline. However, if the concentration is too high, crystal formation will be precluded since a portion of the insulin will form amorphous precipitates that do not give rise to crystals. Thus, stoichiometric quantities are needed to form adsorbed insulin crystals, such that only trace amounts of insulin remain in solution following crystallization and that the insoluble insulin is present as crystals.

A specific protamine concentration (or specific range of applicable concentrations) for a particular insulin, insulin derivative, or insulin analog is empirically determined to be optimal for crystal formation. Typically, the protamine concentration for a particular insulin polypeptide will lie between about 0.25 mg/mL to about 0.32 mg/mL, with the insulin concentration typically lying between 0.57 micromoles/ml to 0.64 micromoles/ml, and preferably being 0.60 micromoles/ml. For example, NPH crystals of human insulin may be prepared at the protamine concentration of 0.31 mg/ml and an insulin concentration of 100 Units/ml, which for human insulin corresponds to a concentration of 0.60 micromoles/ml or 3.5 mg/ml (see Example 2). According to the present invention, the concentration of protamine is determined by precise methods, such as HPLC.

Following the formation of adsorbed insulin crystals in the first step, the adsorbed insulin crystals are converted to non-adsorbed insulin crystals in the second step. This conversion is achieved by quantitatively increasing the protamine content of the crystals at a second, higher protamine concentration. The second protamine concentration is precisely determined, preferably by HPLC, and is chosen to be between 10% to 40% greater than the initial protamine concentration. Accordingly, adsorbed insulin crystals formed from an insulin concentration of about 0.57 micromoles/ml to 0.64 micromoles/ml and an initial protamine concentration of about 0.25 mg/mL to about 0.32 mg/mL correspondingly will be converted to non-adsorbed insulin crystals which have a precisely determined protamine concentration of about 0.28 mg/ml to about 0.45 mg/ml. For example, adsorbed insulin crystals have been formed at an initial protamine concentration of 0.31 mg/ml (and 3.5 mg/ml human insulin) and then subsequently rendered non-adsorbed insulin crystals at a second protamine concentration of 0.36 mg/ml (see Example 2).

Increasing the protamine concentration in a second step may be critical in the formation of non-adsorbed insulin crystals. The higher, second concentration of protamine may be ineffective for use as the first concentration of protamine in the initial formation of adsorbed insulin crystals, since this higher second concentration might give rise to non-crystalline amorphous precipitates. For example, 0.36 mg/ml protamine may be appropriate as the second protamine concentration in the fortification of crystals formed using a particular insulin, insulin derivative, or insulin analog, but may be undesirable as a first protamine concentration in the initial formation of the crystals.

The process of adding a portion of the final amount of protamine after NPH crystals have been formed is herein termed “fortification.” Thus, the non-adsorbed insulin crystals may also be referred to as “fortified NPH.” The non-adsorbed insulin crystals appear to be derived by crystallization of the adsorbed insulin of NPH crystals, as judged by the IAIA and the time action properties of the insulin.

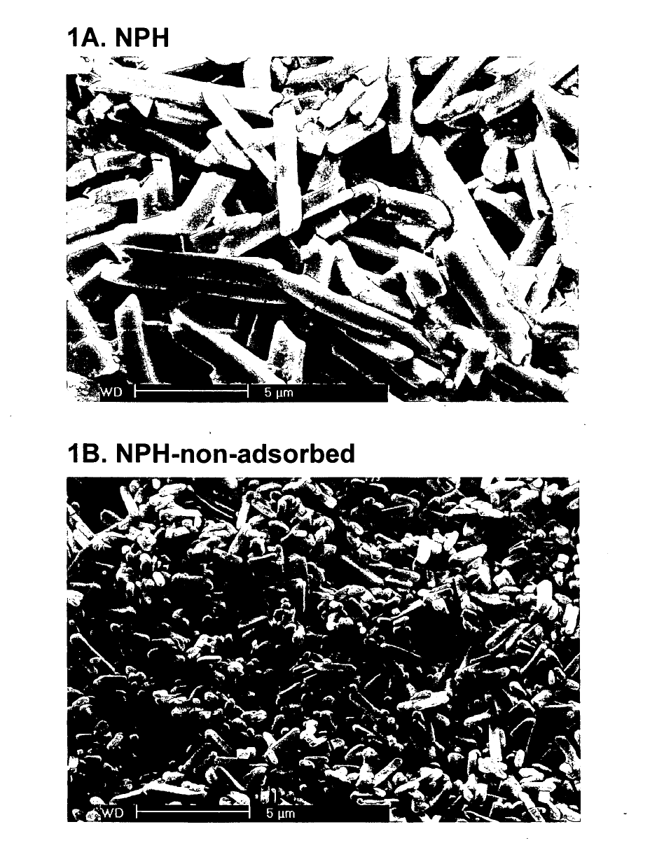

The morphology of the non-adsorbed insulin crystals formed from human insulin has been examined microscopically by optical microscope at 1000× magnification and by scanning electron microscope at 10,000× (see FIG. 1B) and 40,000× magnification. Both adsorbed insulin crystals formed at a first protamine concentration and subsequently formed non-adsorbed insulin crystals possess a uniform, rod-like morphology similar to the well known morphology of NPH crystals. The non-adsorbed insulin crystals preferably are smaller in size than typical NPH crystals, as shown respectively in the scanning electron microscopy images of FIG. 1B (NPH-non-adsorbed) and FIG. 1A (NPH). The non-adsorbed insulin crystals of the invention may vary in size, with the longest dimension of the crystals measuring between 0.5-10 microns, preferably between 0.5-5 microns, and more preferably between 0.5-3 microns. A preparation of crystals of the present invention will contain crystals in which at least 95%, more preferably 97%, and more preferably still 99% of the crystals have a longest dimension within a given preferred range.

These crystal sizes refer to the sizes of single or individual crystals. In solution, the crystals of the present invention may be dispersed as individual crystals, yet typically are dispersed as aggregates or clumps of crystals. One of skill in the art will recognize that clumps of crystals need to be considered during crystal size determination, particularly with respect to size determination by visual microscopy, laser diffraction, and Coulter methodology. Preferably, crystal size is determined by SEM imaging.

The present invention also provides for non-adsorbed insulin crystals that are irregular in morphology. In particular, crystals formed from an insulin derivative or insulin analog may have an irregular morphology. Despite their irregular morphology, the non-adsorbed insulin crystals are crystalline, as opposed to being an amorphous precipitate of insoluble material that is not crystalline in form.

The concept of extending the time-action of insoluble insulin through the incorporation of very high levels of both protamine and zinc are known in the art and are the basis of PZI insulin. The protamine content of PZI insulin is greater than the non-adsorbed insulin crystals of the present invention, as PZI typically contains 370%-560% greater protamine content than NPH, while the present invention contains 10-40% greater protamine content than NPH. Due to its high protamine content, PZI insulin is not mixable with soluble insulin, whereas the non-adsorbed insulin crystals of the present invention are mixable with soluble insulin.

In addition to having higher protamine content than the non-adsorbed crystals of the present invention, PZI insulin also contains greater zinc content. PZI typically contains 6 to 10 times more zinc than the adsorbed insulin crystals disclosed herein. Specifically, PZI contains 150 to 250 micrograms zinc per 100 Units of insulin, whereas insulin crystals of the present invention contain about 25 micrograms zinc per 100 Units of insulin.

A preferred group of insulin analogs for preparing derivatized insulin analogs used to form the present crystalline compositions consists of animal insulins, deletion analogs, and pI-shifted analogs. A more preferred group consists of animal insulins and deletion analogs. Deletion analogs are yet more preferred.

Another preferred group of insulin analogs for use in the crystals of the present invention consists of the rapid acting insulin analogs. Particularly preferred are those rapid acting insulin analogs wherein the amino acid residue at position B3 is Lys or Gly, the amino acid residue at position B28 is Asp, Lys, Leu, Val, or Ala, the amino acid residue at position B29 is Glu, Lys or Pro, the amino acid residue at position B10 is His or Asp, the amino acid residue at position B1 is Phe, Asp or deleted alone or in combination with a deletion of the residue at position B2, the amino acid residue at position B30 is Thr, Ala, Ser, or deleted, and the amino acid residue at position B9 is Ser or Asp; provided that either position B28 or B29 is Lys. Especially preferred is a rapid acting insulin analog selected from the group consisting of LysB28, ProB29-insulin, AspB28-insulin, and LysB3,GluB29-insulin.

Another preferred group of insulin analogs for use in the present invention consists of those wherein the isoelectric point of the insulin analog is between about 7.0 and about 8.0. These analogs are referred to as “pI-shifted insulin analogs.” Examples of pI-shifted insulin analogs include, for example, ArgB31,ArgB32-human insulin, GlyA21,ArgB31,ArgB32-human insulin, ArgA0,ArgB31,ArgB32-human insulin, and ArgA0,GlyA21,ArgB31,ArgB32-human insulin.

Another preferred group of insulin analogs consists of LysB28,ProB29-human insulin (B28 is Lys; B29 is Pro); AspB28-human insulin (B28 is Asp), AspB1-human insulin, ArgB31,ArgB32-human insulin, ArgA0-human insulin, AspB1,GluB13-human insulin, AlaB26-human insulin, GlyA21-human insulin, des(ThrB30)-human insulin, and GlyA21,ArgB31,ArgB32-human insulin.

Especially preferred insulin analogs include LysB28,ProB29-human insulin, des(ThrB30)-human insulin, AspB28-human insulin, and AlaB26-human insulin. Another especially preferred insulin analog is GlyA21, ArgB31, ArgB32-human insulin [Dörschug, M., U.S. Pat. No. 5,656,722, 12 Aug. 1997]. The most preferred insulin analog is LysB28,ProB29-human insulin.

The preferred derivatized proteins are acylated proteins, and the preferred acylated proteins for the crystals and formulations of the present invention are fatty acid-acylated insulin, and fatty acid-acylated insulin analogs. Fatty acid-acylated human insulin is highly preferred. Fatty acid-acylated insulin analogs are equally highly preferred. Examples of such fatty acid-acylated insulin analogs are found in U.S. Pat. No. 6,268,335.

A preferred group of derivatized proteins for use in the insoluble compositions of the present invention consists of mono-acylated proteins. Mono-acylation at the ε-amino group is most preferred. For insulin, mono-acylation at LysB29 is preferred. Similarly, for certain insulin analogs, such as, LysB28,ProB29-human insulin analog, mono-acylation at the ε-amino group of LysB28 is most preferred. Especially preferred mono-acylated insulins are B29-Nε-Octanoyl-human insulin and B29-Nε-Tetradecanoyl-des (B30)-human insulin analog.

The particular group used to derivatize insulin, an insulin analog, or a proinsulin may be any chemical moiety that does not significantly reduce the biological activity of the insulin, is not toxic when bonded to the insulin, and most importantly, reduces the aqueous solubility, raises the lipophilicity, or decreases the solubility of zinc/protamine complexes of the derivatized insulin.

Insulin, insulin analogs, or proinsulins used to prepare derivatized proteins can be prepared by any of a variety of recognized peptide synthesis techniques including classical (solution) methods, solid phase methods, semi-synthetic methods, and more recent recombinant DNA methods. For example, see Chance, R. E., et al., U.S. Pat. No. 5,514,646, 7 May 1996; EPO publication number 383,472, 7 Feb. 1996; Brange, J. J. V., et al. EPO publication number 214,826, 18 Mar. 1987; and Belagaje, R. M., et al., U.S. Pat. No. 5,304,473, 19 Apr. 1994, which disclose the preparation of various proinsulin and insulin analogs.

Generally, derivatized proteins are prepared using methods known in the art. The publications listed above to describe derivatized proteins contain suitable methods to prepare derivatized proteins. Generally, to prepare acylated proteins, the protein is reacted with an activated organic acid, such as an activated fatty acid. The term “activated fatty acid ester” means a fatty acid which has been activated using general techniques known in the art [Riordan, J. F. and Vallee, B. L., Methods in Enzymology, XXV:494-499 (1972); Lapidot, Y., et al., J. Lipid Res. 8:142-145 (1967)]. Hydroxybenzotriazide (HOBT), N-hydroxysuccinimide and derivatives thereof are particularly well known for forming activated acids for peptide synthesis.

Aqueous compositions containing water as the major solvent are preferred. Aqueous suspensions wherein water is the solvent are highly preferred.

The compositions of the present invention are used to treat patients who have diabetes or hyperglycemia. Accordingly, the non-adsorbed insulin crystals of the present invention may be used for the manufacture of a medicament for the treatment of diabetes mellitus or hyperglycemia.

Formulations of the non-adsorbed insulin crystals of the present invention will typically provide insulin at concentrations of from about 1 mg/mL to about 10 mg/mL. Present formulations of insulin products are typically characterized in terms of the concentration of units of insulin activity (units/mL), such as U40, U50, U100, and so on, which correspond roughly to about 1.4, 1.75, and 3.5 mg/mL preparations, respectively. The dose, route of administration, and the number of administrations per day will be determined by a physician considering such factors as the therapeutic objectives, the nature and cause of the patient's disease, the patient's gender and weight, level of exercise, eating habits, the method of administration, and other factors known to the skilled physician. In broad range, a daily dose would be in the range of from about 1 nmol/kg body weight to about 6 nmol/kg body weight (6 nmol is considered equivalent to about 1 unit of insulin activity). A dose of between about 2 and about 3 nmol/kg is typical of present insulin therapy.

The physician of ordinary skill in treating diabetes will be able to select the therapeutically most advantageous means to administer the formulations of the present invention. Parenteral routes of administration are preferred. Typical routes of parenteral administration of suspension formulations of insulin are the subcutaneous and intramuscular routes. The compositions and formulations of the present invention may also be administered by nasal, buccal, pulmonary, or occular routes.

Glycerol at a concentration of 12 mg/mL to 25 mg/mL is preferred as an isotonicity agent. Yet more highly preferred for isotonicity is to use glycerol at a concentration of from about 15 mg/mL to about 17 mg/mL.

M-cresol and phenol, or mixtures thereof, are preferred preservatives in formulations of the present invention.

For efficient yield of crystals, the molar ratio of zinc to total protein in the crystal of the present invention is bounded at the lower limit by about 0.33, that is, the approximately two zinc atoms per hexamer which are needed for efficient hexamerization. The crystal and amorphous precipitate compositions will form suitably with about 2 to about 4-6 zinc atoms present when no compound that competes with insulin for zinc binding is present. Even more zinc may be used during the process if a compound that competes with the protein for zinc binding, such as one containing citrate or phosphate, is present. Excess zinc above the minimum amount needed for efficient hexamerization may be desirable to more strongly drive hexamerization. Also, excess zinc above the minimum amount can be present in a formulation of the present invention, and may be desirable to improve chemical and physical stability, to improve suspendability, and possibly to further extend time-action. Consequently, there is a fairly wide range of zinc:protein ratios allowable in the insoluble compositions, processes, and formulations of the present invention.

Accordingly, zinc is present in the formulation in an amount of from about 0.3 mole to about 7 moles per mole of total insulin and more preferably about from 0.3 mole to about 1.0 mole per mole of total insulin. For a derivatized insulin, a highly preferred ratio of zinc to derivatized insulin is from about 0.3 to about 0.7 mole of zinc atoms per mole of total insulin. Most highly preferred is a ratio of zinc to total insulin from about 0.30 to about 0.55 mole of zinc atoms per mole of total insulin.

The zinc compound that provides zinc for the present invention may be any pharmaceutically acceptable zinc compound. The addition of zinc to insulin preparations is known in the art, as are pharmaceutically acceptable sources of zinc. Preferred zinc compounds to supply zinc for the present invention include zinc chloride, zinc acetate, zinc citrate, zinc oxide, and zinc nitrate.

Protamine is used in the present invention to precipitate and subsequently crystallize hexamers of insulin. Protamine is present in the non-adsorbed insulin crystal in an amount of from about 0.3 mg/ml to about 0.48 mg/ml, with insulin being present at about 3.3 mg/ml to about 3.7 mg/ml. For example, the non-adsorbed insulin crystals presented in Example 2 contain 0.36 mg/ml protamine and 3.5 mg/ml (100 Units/ml) human insulin. Protamine sulfate is the preferred salt form for use in the present invention.

Another component of the crystals of the present invention is a hexamer stabilizing compound, which may be any of a wide range of suitable compounds. Preferred hexamer stabilizing compounds include phenol and m-cresol. They must be present in sufficient proportions with respect to total protein to stabilize the desired conformation. To accomplish this, at least 2 or at least 3 moles of hexamer stabilizing compound per hexamer are required for effective hexamer stabilization. It is preferred that at least 3 moles of hexamer stabilizing compound per hexamer be present in the crystals and precipitates of the present invention. The presence of higher ratios of hexamer stabilizing compound, at least up to 25 to 50-fold higher, in the solution from which the crystals are prepared will not adversely affect hexamer stabilization. Preferred hexamer stabilizing compounds include the phenolic compounds phenol and m-cresol, as well as mixtures of these compounds. For example, a preferable phenolic mixture will contain 0.72 mg/ml phenol and 1.76 mg/ml m-cresol.

The non-adsorbed insulin crystals of the present invention are typically formed in the presence of a buffer, such as citrate, phosphate, acetate, TRIS, and glycine. Preferably, the buffer used in the formation of these crystals is citrate.

In formulations of the present invention, a preservative may be present, especially if the formulation is intended to be sampled multiple times. As mentioned above, a wide range of suitable preservatives are known. Preferably, the preservative is present in the solution in an amount suitable to provide an antimicrobial effect sufficient to meet pharmacopoeial requirements. Where appropriate, the preservative may be the same compound(s) used as the hexamer-stabilizing compound(s).

Preferred preservatives are the phenolic preservatives. Preferred concentrations for the phenolic preservative are from about 2 mg to about 5 mg per milliliter of the aqueous suspension formulation. These concentrations refer to the total mass of phenolic preservatives because mixtures of individual phenolic preservatives are contemplated. Suitable phenolic preservatives include, for example, phenol, m-cresol, and methylparaben. Preferred phenolic compounds are phenol and m-cresol. Mixtures of phenolic compounds, such as phenol and m-cresol, are also contemplated and highly preferred. Examples of mixtures of phenolic compounds are 0.6 mg/mL phenol and 1.6 mg/mL m-cresol, and 0.7 mg/mL phenol and 1.8 mg/mL m-cresol.

The present invention provides processes for preparing the non-adsorbed insulin compositions. Also, the use of the present insoluble compositions to prepare medicaments for controlling blood glucose, and for treating diabetes or hyperglycemia is contemplated. Processes for preparing the non-adsorbed insulin compositions will typically have two overall steps: (1) formation of adsorbed insulin crystals of protamine, zinc, insulin and a hexamer-stabilizing compound at a precise first concentration of protamine, and (2) formation of non-adsorbed insulin crystals by quantitatively increasing the protamine content of the crystals formed in step (1) to a precise second concentration of protamine.

The overall first step of formation of adsorbed insulin crystal is analogous to conventional preparation of insulin crystals, which is well known in the art (see, for example, U.S. Pat. No. 6,268,335). A critical aspect of the present invention is the precise quantitation of protamine by methods such as HPLC, which both enables optimal crystal formation and the subsequent, second step of precisely increasing the protamine concentration.

After the formation of adsorbed insulin crystals is completed, the majority of the starting insulin will be associated with crystals, either as crystallized or adsorbed insulin. However, the supernatant of the crystallization solution will still typically contain low amounts of soluble insulin. This soluble insulin will typically represent between about 0.4% to about 0.8%, even upwards of 1% of the total starting amount of insulin.

In the second step of producing non-adsorbed crystals, the protamine content of the adsorbed insulin crystals is increased to yield non-adsorbed insulin crystals. In this fortification process, the amount of protamine in the solution is quantitatively increased by 10% to 40% over that used in the first step formation of adsorbed insulin crystals. The concentration of protamine is quantitated using a precise method such as HPLC. Following formation of non-adsorbed insulin crystals, the supernatant of the solution will contain very low amounts of soluble insulin, which will typically be about 0% to about 0.2% of the total starting amount of insulin.

After the non-adsorbed insulin crystals of the present invention are formed, they may be separated from the remaining solution components and introduced into a different aqueous solvent or medium, for storage and administration to a patient. Examples of appropriate aqueous solvents are as follows: water for injection containing 25 mM TRIS, 5 mg/mL phenol and 16 mg/mL glycerol; water for injection containing 2 mg/mL sodium phosphate dibasic, 1.6 mg/mL m-cresol, 0.65 mg/mL phenol, and 16 mg/mL glycerol; and water for injection containing 25 mM TRIS, 5 mg/mL phenol, 0.01 M trisodium citrate, and 16 mg/mL glycerol.

In a preferred embodiment, the crystals are prepared in a manner that obviates the need to separate the crystals from the remaining solution components. Thus, it is preferred that the solution itself be suitable for administration to the patient, or that the solution can be made suitable for administration by dilution with a suitable, pharmaceutically acceptable diluent. The term pharmaceutically acceptable diluent will be understood to mean a solution comprised of an aqueous solvent in which is dissolved various pharmaceutically acceptable excipients, including without limitation, a buffer, an isotonicity agent, zinc, a preservative, protamine, and the like.

In addition to insulin, zinc, protamine, and hexamer-stabilizing compound, pharmaceutical compositions adapted for parenteral administration in accordance with the present invention may employ additional excipients and carriers such as water miscible organic solvents such as glycerol, sesame oil, aqueous propylene glycol and the like. When present, such agents are usually used in an amount less than about 2.0% by weight based upon the final formulation. For further information on the variety of techniques using conventional excipients or carriers for parenteral products, please see Remington's Pharmaceutical Sciences, 17th Edition, Mack Publishing Company, Easton, Pa., USA (1985), which is incorporated herein by reference.

In the practice of the present invention, it is also contemplated that a formulation may contain a mixture of the non-adsorbed insulin crystals and a soluble fraction of a protein selected from insulin, derivatized insulin, insulin analogs, and derivatized insulin analogs. Examples of such pharmaceutical compositions include sterile, isotonic, aqueous saline solutions of insulin, an insulin analog, a derivatized insulin, or a derivatized insulin analog, buffered with a pharmaceutically acceptable buffer and pyrogen-free. Preferred for the soluble phase are insulin or a rapid-acting insulin analog, such as, LysB28,ProB29-human insulin, AspB28-human insulin, or LysB3,GluB29-human insulin. Such mixtures are designed to provide a combination of meal-time control of glucose levels, which is provided by the soluble insulin, and basal control of glucose levels, which is provided by the insoluble insulin. The ratio of total protein in the insoluble phase and total protein in the soluble phase is in the range of about 9:1 to about 1:9. A preferred range of this ratio is from about 9:1 to about 1:1, and more preferably, about 7:3. Other ratios are 1:1, and 3:7.

In the examples described below, amounts of protamine and insulin were analyzed by reversed-phase gradient HPLC. Briefly, the analytical system relied on a C8 reversed-phase column, at 23° C. The flow rate was 1.0 mL/min and UV detection at 214 nm was used. Solvent A was 0.1% (vol:vol) trifluoroacetic acid (TFA) in 10:90 (vol:vol) acetonitrile:water. Solvent B was 0.12% (vol:vol) TFA in 90:10 (vol:vol) acetonitrile:water. The gradient was (minutes, % B): (0.1,0); (45.1,75); (50.1,100); (55,100); (57,0); (72,0). All changes were linear. Other analytical systems could be devised by the skilled person to achieve the same objective.

HPLC was used to determine protein concentrations. The retention times of peaks in the chromatograms of protamine and proteins obtained from insulin crystals were compared with the retention times observed for protamine and the active compounds used to make the formulations. Concentrations of protein were determined by comparing the appropriate peak areas to the areas of a standard. A 0.22 mg/mL solution of insulin is typically used as the standard for insulin. A solution of protamine having a specific concentration within the range of 0.05 to 0.1 mg/mL is used as the protamine standard.

The following examples illustrate and explain the invention. The scope of the invention is not limited to these examples.

### EXAMPLE 1

Immediately Available Insulins Assay (IAIA)

A solution of 0.1 M Tris buffer is prepared. To prepare 500 mL of the buffer, 3.54 g of Tris-HCl and 3.34 g of Tris-base are dissolved and diluted with water to 500 mL in a volumetric flask. The pH value of the resulting solution is checked on the day of the assay and must be between 8.15 and 8.35.

A sample of the crystal formulation for analysis is resuspended by gentle agitation and 2.00 mL is combined with 2.00 mL of Tris buffer. This preparation is swirled occasionally to keep suspended. Ten minutes after combining the formulation and tris buffer, the mixture is filtered through a 0.2 micron low protein-binding filter. 2.00 mL of the filtrate is added to a 5 mL volumetric flask; 1 mL of 0.2N HCl is then added. Then the solution is diluted to 5.00 mL with 0.01N HCl to produce the solution for HPLC analysis.

The reversed phase HPLC method utilizes a Waters column (WAT094263) at room temperature. A Hewlett-Packard autoinjector with a refrigerated sample tray set for an injection volume of 100 microliters is used.

Mobile Phase:

Solution A=10% acetonitrile, 90% water, 0.1% trifluoroacetic acid
Solution B=90% acetonitrile, 10% water, 0.12% trifluoroacetic acid
Flow: 1.5 mL/min
Detection wavelength=214 nm

Gradient Used with Beckman 126 Pumping System:

| Time (min.) | % B | Duration (min) |
|---|---|---|
| 0 | 0 | — |
| 0.1 | 38 | 10 |
| 10.1 | 65 | 2 |
| 12.1 | 100 | 1 |
| 15 | 0 | 0.1 |
| 18 | End | — |

Solutions containing known concentrations of insulin are used to generate a standard curve. This standard curve is used to determine the immediately available insulin concentration of the formulation.

### EXAMPLE 2

Preparation of Protamine Fortified Insulin Suspension

Insulin solution was prepared as follows. 611.5 mg of zinc insulin was dissolved in 16 mL of 0.1 N HCl. To this solution was added 0.155 mL of an accurately determined 10 mg/mL zinc solution (prepared by dissolving an accurately weighed quantity of ZnO in HCl).

A buffer solution was prepared comprising 45.71 mg/mL glycerol, 4.20 mg/mL trisodium citrate dihydrate, 2.06 mg/mL phenol, 5.03 mg/mL meta-cresol, and 10.71 mg/mL dibasic sodium phosphate heptahydrate at pH 8.3.

The zinc insulin solution was added to 56 mL of the buffer solution, and the pH was adjusted to 7.6 with the addition of 0.15 mL 5 N NaOH. Sterile water was then added to bring the total volume to 80 mL.

A protamine solution was prepared by dissolving protamine sulfate in water; the protamine concentration was determined to be 0.6567 mg/mL by HPLC. An aliquot was diluted to 0.62 mg/mL with water and filtered through an 0.2 micron Acrodisc 4496 filter.

To prepare an insulin-protamine suspension, 25 mL of the 0.62 mg/mL protamine solution was added to 25 mL of the insulin solution. A precipitate formed immediately which was stirred for 5 minutes, then placed in a temperature-controlled water bath at 25 degrees C.

After about 27 hours, a small sample was removed for inspection under an optical microscope at 1000× magnification. The precipitate had formed uniform crystals possessing a rod-like shape. These uniform, adsorbed insulin crystals were then used to prepare non-adsorbed insulin crystals by increasing the protamine concentration of the formulation.

A protamine solution for increasing the concentration of protamine in the insulin-protamine suspension was prepared by dissolving protamine sulfate in water. The protamine concentration was determined accurately by HPLC as 9.45 mg/mL.

To increase the concentration of protamine in the insulin-protamine suspension, the insulin-protamine suspension was first stirred gently to resuspend the solids. To 25 mL of this stirred suspension was added 0.132 mL of the 9.45 mg/mL protamine solution, followed by stirring for 30 minutes. This fortification increased the total protamine content of the suspension from 0.31 mg/mL to 0.36 mg/mL.

The final target concentrations in the formulation:

| Insulin | 3.5 mg/mL |
|---|---|
| Zinc | 0.025 mg/mL |
| Dibasic Sodium Phosphate heptahydrate | 3.75 mg/mL |
| Meta-cresol | 1.76 mg/mL |
| Phenol | 0.72 mg/mL |
| Trisodium citrate dihydrate | 1.47 mg/mL |
| Glycerin | 16 mg/mL |
| Protamine | 0.36 mg/mL |
| pH | 7.2-8.0 |

In the formulation for the adsorbed insulin crystals, the target concentrations were the same for each component except protamine, which was present at 0.31 mg/ml.

The non-adsorbed insulin crystals of the formulation have 0.1-0.2% of immediately available insulin as determined by the IAIA. The adsorbed insulin crystals of the formulation prior to fortification have 3-4% of immediately available insulin. The decrease in the amount of immediately available insulin after fortification is attributed to the decrease in adsorbed insulin on the crystals.

The formulation is stored according to typical NPH storage guidelines, such as those which specify storage in a cool place without freezing. This formulation is administered to a patient as a long-acting insulin for the treatment of diabetes.

### EXAMPLE 3

Preparation of Protamine Fortified Insulin Analog Suspension

LysB28,ProB29-human insulin analog solution is prepared as follows. 611.5 mg of zinc LysB28,ProB29-human insulin analog insulin is dissolved in 16 mL of 0.1 N HCl. To this solution is added) 0.155 mL of an accurately determined 10 mg/mL zinc solution (prepared by dissolving an accurately weighed quantity of ZnO in HCl).

A buffer solution is prepared comprising 45.71 mg/mL glycerol, 4.20 mg/mL trisodium citrate dihydrate, 2.06 mg/mL phenol, 5.03 mg/mL meta-cresol, and 10.71 mg/mL dibasic sodium phosphate heptahydrate at pH 8.3.

The zinc insulin analog solution is added to 56 mL of the buffer solution, and the pH is adjusted to 7.6 with the addition of 0.15 mL 5 N NaOH. Sterile water is then added to bring the total volume to 80 mL.

A protamine solution having a concentration greater than 0.62 mg/ml is prepared by dissolving protamine sulfate in water; the protamine concentration is determined by HPLC. An aliquot is diluted to 0.62 mg/mL with water and filtered through an 0.2 micron Acrodisc 4496 filter.

To prepare an insulin analog-protamine suspension, 25 mL of the 0.62 mg/mL protamine solution is added to 25 mL of the insulin solution. These two solutions are equilibrated at 15 degrees C. prior to mixing. A precipitate forms immediately and is stirred for 5 minutes, and is then placed in a temperature-controlled water bath at 15 degrees C.

After about 24 hours, a small sample is removed for inspection under an optical microscope at 1000× magnification. The precipitate will form uniform crystals possessing a rod-like shape, much like NPH. These adsorbed insulin crystals are then used to prepare non-adsorbed insulin crystals by increasing the protamine concentration of the formulation.

A second protamine solution for increasing the concentration of protamine in the insulin-protamine suspension is prepared by dissolving protamine sulfate in water. The protamine concentration is between about 9.4 mg/ml to about 10.0 mg/ml, and is determined accurately by HPLC.

To increase the concentration of protamine in the insulin-protamine suspension, the insulin-protamine suspension is first stirred gently to resuspend the solids. To 25 mL of this stirred suspension is added an appropriate amount of the second protamine solution to increase the total protamine content of the suspension from 0.31 mg/mL to 0.36 mg/mL, followed by stirring for 30 minutes.

The final target concentrations in the formulation:

| Insulin analog | 3.5 mg/mL |
|---|---|
| Zinc | 0.025 mg/mL |
| Dibasic Sodium Phosphate heptahydrate | 3.75 mg/mL |
| Meta-cresol | 1.76 mg/mL |
| Phenol | 0.72 mg/mL |
| Trisodium citrate dihydrate | 1.47 mg/mL |
| Glycerin | 16 mg/mL |
| Protamine | 0.36 mg/mL |
| pH | 7.2-8.0 |

In the formulation for the adsorbed insulin crystals, the target concentrations are the same for each component except protamine, which is present at 0.31 mg/ml.

The non-adsorbed insulin crystals of the formulation have 0.1-0.2% of immediately available insulin as determined by the IAIA. The adsorbed insulin crystals of the formulation prior to fortification have 3-4% of immediately available insulin. The decrease in the amount of immediately available insulin after fortification is attributed to the decrease in adsorbed insulin on the crystals.

The formulation is stored according to typical NPH storage guidelines, such as those which specify storage in a cool place without freezing. This formulation is administered to a patient as a long-acting insulin for the treatment of diabetes.

### EXAMPLE 4

Preparation of Protamine Fortified Insulin Suspension from Commercial NPH.

A sample of commercial NPH is assayed using the precise HPLC method of protamine determination, as described in Example 1. To this commercial NPH (such as a 10 mL vial containing U100) is added a small volume of an accurately HPLC quantitated protamine stock solution (e.g. 10 mg/mL in water) to bring the final total concentration of protamine to 0.36 mg/mL. This sample is stirred gently for 30 minutes, then stored according to typical NPH storage guidelines.

The commercial NPH has 3 to 4% of immediately available insulin as determined by the IAIA. Following fortification, the formulation contains 0.1 to 0.5% of immediately available insulin. The decrease in the amount of immediately available insulin after fortification is attributed to the decrease in adsorbed insulin on the crystals.

The formulation is administered to a patient as a long-acting insulin for the treatment of diabetes.

### EXAMPLE 5

In Vivo Testing in Diabetic Dogs

The protracted action of a suspension formulation containing non-adsorbed insulin crystals prepared as described herein is tested in diabetic dogs by comparing its ability to control hyperglycemia with that of control compounds. On test days, blood glucose is monitored for 24 hours following subcutaneous injection of the suspension formulation.

Specifically, the time-action of compositions of the present invention is determined in normal dogs that received a constant infusion of somatostatin to create a transient diabetic state. A non-adsorbed insulin crystal formulation, comprising human insulin, is prepared essentially as described in Example 2, and is administered subcutaneously at a dose of 2 nmol/kg. The data is compared to that observed in the same model after administration of Humulin N (2.0 nmol/kg “NPH”), Beef/Pork Ultralente insulin (3 nmol/kg, “BP-UL”), and saline.

Experiments are conducted in overnight-fasted, chronically cannulated, conscious male and female beagles weighing 10-17 kg (Marshall Farms, North Rose, N.Y.). At least ten days prior to the study, animals are anesthetized with isoflurane (Anaquest, Madison, Wis.), and silicone catheters attached to vascular access ports (V-A-P™, Access Technologies, Norfolk Medical, Skokie, Ill.) are inserted into the femoral artery and femoral vein. The catheters were filled with a glycerol/heparin solution (3:1, v/v; final heparin concentration of 250 KIU/mL; glycerol from Sigma Chemical Co., St. Louis, Mo., and heparin from Elkins-Sinn, Inc., Cherry Hill, N.J.) to prevent catheter occlusion, and the wounds are closed. Kefzol (Eli Lilly & Co., Indianapolis, Ind.) is administered pre-operatively (20 mg/kg, IV and 20 mg/kg, I.M.), and Keflex is administered post-operatively (250 mg, p.o. once daily for seven days) to prevent infections. Torbugesic (1.5 mg/kg, I.M.) is administered post-operatively to control pain.

Blood is drawn just prior to the study day to determine the health of the animal. Only animals with hematocrits above 38% and leukocyte counts below 16,000/mm³ are used (hematology analyzer: Cell-Dyn 900, Sequoia-Turner, Mountain View, Calif.).

The morning of the experiment, the ports are accessed (Access Technologies, Norfolk Medical, Skokie, Ill.); the contents of the catheters are aspirated; the catheters are flushed with saline (Baxter Healthcare Corp., Deerfield, Ill.); the dog is placed in a cage; and extension lines (protected by a stainless steel tether and attached to a swivel system [Instech Laboratories, Plymouth Meeting, Pa.]) were attached to the port access lines.

Dogs are allowed at least 10 minutes to acclimate to the cage environment before an arterial blood sample was drawn for determination of fasting insulin, glucose, and glucagon concentrations (time=−30 minutes). At this time, a continuous, IV infusion of cyclic somatostatin (0.65 μg/kg/min; BACHEM California, Torrance, Calif.) is initiated and continued for the next 30.5 hours. Thirty minutes after the start of infusion (time=0 minutes), an arterial blood sample is drawn, and a subcutaneous bolus of test substance, or vehicle, is injected in the dorsal aspect of the neck. Arterial blood samples are taken every 3 hours thereafter for the determination of plasma glucose and insulin concentrations and every 6 hours for determination of plasma glucagon concentrations. The entire study lasts 30 hours.

Arterial blood samples are collected in vacuum blood collection tubes containing disodium EDTA (Terumo Medical Corp., Elkton, Md.) and immediately placed on ice. A portion of the blood sample (1.5 mL) is transferred to a polypropylene tube containing 40 μl of aprotinin (10,000 KIU/mL; Trasylol, Miles, Inc., Diagnostics Division, Kankakee, Ill.) in preparation for the determination of the plasma glucagon concentration. The samples are centrifuged, and the resulting plasma is transferred to polypropylene test tubes and stored on ice for the duration of the study.

Plasma glucose concentrations are determined the day of the study using glucose oxidase with a commercial glucose analyzer. Samples for other assays are stored at −80° C. until time for analysis. Insulin concentrations are determined using a double antibody radioimmunoassay. Glucagon concentrations are determined using a radioimmunoassay kit (LINCO Research, Inc., St. Charles, Mo.).

At the conclusion of the experiment, the catheters are flushed with fresh saline, treated with Kefzol (20 mg/kg), and filled with the glycerol/heparin mixture; antibiotic (Keflex; 250 mg) is administered p.o. To minimize the number of animals being used and to allow pairing of the data base when possible, animals are studied multiple times. Experiments in animals being restudied are carried out a minimum of one week apart.

Suspension formulations of non-adsorbed insulin crystals of the present invention may reduce blood glucose levels and may have an extended time action compared with human insulin NPH when tested at comparable doses.

All patents, patent applications, articles, books and other publications cited herein are incorporated by reference in their entireties.

1. Non-adsorbed insulin crystals comprising:
(a) a polypeptide selected from the group consisting of insulin, an insulin analog, and a derivatized insulin;
(b) zinc, present at about 0.3 mole to about 1 mole per mole of polypeptide;
(c) protamine; and
(d) a hexamer-stabilizing compound,
wherein less than 2% of said polypeptide is present on said non-adsorbed insulin crystals as adsorbed polypeptide, and wherein said non-adsorbed crystals have a longest dimension that is between 0.5 to 10 microns.

2. The crystals of claim 1, wherein less than 1% of said polypeptide is present on said non-adsorbed insulin crystals as adsorbed polypeptide.

3. The crystals of claim 1, wherein less than 0.2% of said polypeptide is present on said non-adsorbed insulin crystals as adsorbed polypeptide.

4. The crystals of claim 1, wherein said protamine is present at about 0.29 mg/ml to about 0.45 mg/ml per 0.57 micromoles/ml to 0.64 micromoles/ml of said polypeptide.

5. The crystals of claim 1, wherein said polypeptide is human insulin.

6. The crystals of claim 1, wherein said polypeptide is a derivatized insulin.

7. The crystals of claim 6, wherein said derivatized insulin is an acylated insulin.

8. The crystals of claim 7, wherein said acylated insulin is B29-Nε-octanoyl-human insulin.

9. The crystals of claim 7, wherein said acylated insulin is B29-Nε-tetradecanoyl-des(B30)-human insulin.

10. The crystals of claim 1, wherein said polypeptide is an insulin analog selected from the group consisting of ArgB31,ArgB32-human insulin, and GlyA21,ArgB31,ArgB32-human insulin.

11. The crystals of any of claims 1-10, wherein said non-adsorbed crystals have a longest dimension that is between 0.5 to 5 microns.

12. The crystals of any of claims 1-10, wherein said non-adsorbed crystals have a longest dimension that is between 0.5 to 3 microns.

13. A method of preparing non-adsorbed insulin crystals, said method comprising:
(a) crystallizing ingredients comprising (i) a polypeptide selected from the group consisting of insulin, an insulin analog, and a derivatized insulin, (ii) zinc, present at about 0.3 mole to about 1 mole per mole of polypeptide, (iii) a first concentration of protamine, and (iv) a hexamer-stabilizing compound to form adsorbed insulin crystals; and
(b) combining said adsorbed insulin crystals with protamine so as to achieve a second, higher concentration of protamine to form said non-adsorbed insulin crystals, wherein less than 2% of said polypeptide is present on said non-adsorbed insulin crystals as adsorbed polypeptide, and wherein said non-adsorbed crystals have a longest dimension that is between 0.5 to 10 microns.

14. The method of claim 12, wherein less than 1% of said polypeptide is present on said non-adsorbed insulin crystals as adsorbed polypeptide.

15. The method of claim 12, wherein less than 0.2% of said polypeptide is present on said non-adsorbed insulin crystals as adsorbed polypeptide.

16. The method according to claim 13, wherein said first concentration of protamine is about 0.25 mg/ml to about 0.32 mg/ml per 0.57 micromoles/ml to 0.64 micromoles/ml of said polypeptide, and said second concentration of protamine is about 10% to about 40% greater than said first concentration of protamine.

17. The method according to claim 13, wherein said polypeptide is human insulin.

18. The method according to claim 13, wherein said polypeptide is a derivatized insulin.

19. The method according to claim 18, wherein said derivatized insulin is an acylated insulin.

20. The method according to claim 19, wherein said acylated insulin is B29-Nε-octanoyl-human insulin.

21. The method of claim 20, wherein said acylated insulin is B29-Nε-Tetradecanoyl-des(B30)-human insulin.

22. The method according to claim 13, wherein said polypeptide is an insulin analog selected from the group consisting of ArgB31,ArgB32-human insulin, GlyA21,ArgB31,ArgB32-human insulin.

23. The method according to claim 13, wherein said ingredients further comprise a buffer selected from the group consisting of citrate, phosphate, acetate, TRIS, and glycine.

24. The method according to claim 23, wherein said buffer is citrate.

25. The method according to any of claims 13-24, wherein said non-adsorbed crystals have a longest dimension that is between 0.5 to 5 microns.

26. The crystals of any of claims 13-24, wherein said non-adsorbed crystals have a longest dimension that is between 0.5 to 3 microns.

27. A composition comprising the crystals of claim 1.

28. A pharmaceutical composition comprising the crystals of claim 1 and a pharmaceutically acceptable excipient.

29. A method of treating diabetes mellitus comprising administering the non-adsorbed insulin crystals of claim 1 to a patient in need thereof in a quantity sufficient to regulate blood glucose levels in the patient.

30. The method of claim 29, wherein said quantity provides an insulin effect from about 8 hours to about 24 hours after administration.

31. The method of claim 29, wherein said quantity provides an insulin effect from about 10 hours to about 24 hours after administration.

32. The method of claim 29, wherein said quantity provides an insulin effect from about 12 hours to about 24 hours after administration.
